(19) United States
(12) Patent Application Publication
(10) Pub. No.: US 2012/0097886 A1
(43) Pub. Date: Apr. 26, 2012

(54) NANOCOMPOSITES INCLUDING CARBON NANOTUBES HAVING METAL NANOPARTICLES

(76) Inventors: Sundara RAMAPRABHU, Chennai (IN); Neetu JHA, Bangalore (IN)

(21) Appl. No.: 12/909,547

(22) Filed: Oct. 21, 2010

(51) Int. Cl. C09K 5/00; C07F 15/00; C07F 1/10; C07F 1/12; B82Y 5/00

(52) U.S. Cl. 252/74; 556/112; 556/136; 977/746; 977/748; 977/752

(57) ABSTRACT

Compositions include a multi-walled nanotube including metal nanoparticles. The metal nanoparticles are bound to the multi-walled nanotube through functional groups on a surface of the multi-walled nanotube.

The present technology generally relates to nanofluids comprising multi-walled nanotubes.

### BACKGROUND

Most traditional heat transfer fluids have a poor heat transfer rate. Carbon nanotubes (CNT), especially multi-walled nanotubes (MWNT), are known to have a very high thermal conductivity. These thermally conductive nanomaterials may therefore be used as additives in coolant fluids to enhance the coolant properties, for example. Such fluids where nanomaterials are dispersed in heat-transfer fluids or base fluids are referred to as nanofluids. Without being bound by theory, it is believed that the dispersion of nanomaterials in a base fluid leads to a large enhancement in thermal conductivity of the suspension due to the small size of the nanoparticles. In addition, larger surface area nanoparticles increase the stability and reduce the sedimentation problem of fluid suspensions.

Recently, there has been increased interest in using nanomaterials in nanofluids. Some of the problems associated with metal nanoparticles dispersed in base fluids include, but are not limited to: (a) one has to disperse a large amount of the materials which may lead to settlement of the nanoparticles instead of dispersion; (b) aggregation of nanoparticles which also leads to enhancement in viscosity, which is highly undesirable for fluid flow as a coolant, and which takes place at volume fractions as low as 0.2%; and (c) the use of surfactants or dispersants to disperse the nanoparticles in base fluids is necessary. In the absence of a surfactant or a stabilizer, almost all nanoparticles are unstable in the base fluid because of their high surface energy.

Carbon nanotubes (CNT) have excellent thermal conductivity. One of the main obstacles for the use of CNTs in a nanofluid is the hydrophobic nature of the CNTs. This hydrophobic nature can be attributed to a high cohesive energy of greater than 0.5 eV/nm. Such high cohesive energy arises from tube to tube van der Waal interactions. This cohesive energy is significantly larger than the solvation energy. For the dispersion of MWNTs in polar solvents, it is necessary to impart a hydrophilic nature which can be achieved by oxidatively modifying their outer graphitic surfaces.

Many approaches have been attempted to solubilize CNTs such as covalent and non-covalent functionalization, chemical oxidation, adding of surfactants and attachment of several functional groups on the surface. While these methods are sufficient for dispersion, chemical reagents are needed to make solubilization in the base fluid possible.

### SUMMARY

In one aspect, a composition which includes a multi-walled nanotube including metal nanoparticles is provided. In some embodiments, the metal nanoparticles are bound to the multi-walled nanotube through functional groups on a surface of the multi-walled nanotube.

In some embodiments, the metal nanoparticle includes a transition metal. In some such embodiments, the transition metal is Au, Ag, Pd, Pt, Cu, Ni, Co, Rh, or Ir. In some embodiments, the functional group is a carboxylate, hydroxyl, or carbonyl group.

In another aspect, a nanofluid is provided, where the nanofluid includes a composition containing metal nanoparticles bound to MWNTs through functional groups on the surface of the nanotube, dispersed in a base fluid, however such nanofluids do not include a surfactant or dispersion agent. In some embodiments, the base fluid is de-ionized water. In other embodiments, the base fluid is ethylene glycol.

In some embodiments, the nanofluid exhibits a thermal conductivity that is at least 5% greater than the base fluid without the composition. In other embodiments, the nanofluid exhibits a thermal conductivity that is at least 25% greater than the base fluid without the composition.

In some embodiments, the nanofluid includes a multi-walled nanotube coated with Pd, Au, or Ag; and a base fluid that is water or ethylene glycol, however such nanofluids do not include a surfactant or dispersion agent.

In another aspect, a method of preparing a composition which includes a multi-walled nanotube including metal nanoparticles, is provided. The method includes refluxing the multi-walled nanotube in concentrated acid to form a multi-walled nanotube which includes functional groups, collecting the multi-walled nanotube comprising functional groups,

reacting the multi-walled nanotube which includes functional groups, a metal salt, and a reductant in a solvent to form the multi-walled nanotube including metal nanoparticles.

In some embodiments, the metal salt is a transition metal salt. In some embodiments, the transition metal salt includes Au, Ag, Pd, Pt, Cu, Ni, Co, Rh, or Ir. In some embodiments, the metal salt includes F⁻; Cl⁻; Br⁻; I⁻; NO₃⁻; CN⁻; ClO₄⁻; or CH₃COO⁻.

The reductant can be any suitable reducing agent known in the art. In some embodiments, the reductant is NaBH₄. The solvent used for reduction process can be any suitable solvent in which the functionalized MWNTs can be effectively dispersed and the metal salts can be solubilized. In one embodiment, the solvent is acetone.

In another aspect, a method of preparing a nanofluid is provided, where the nanofluid includes a composition containing metal nanoparticles bound to MWNTs through functional groups on the surface of the nanotube, dispersed in a base fluid, however, such nanofluids do not include a surfactant or dispersion agent. The methods include sonicating the multi-walled nanotube including metal nanoparticles in a base fluid. In some embodiments, the base fluid includes water or ethylene glycol.

In some embodiments, the nanofluid includes multi-walled nanotubes including Pd, Au, or Ag nanoparticles and the base fluid includes water or ethylene glycol.

### BRIEF DESCRIPTION OF THE DRAWINGS

FIG. 1 is a schematic representation of metal-MWNTs as prepared from functionalized MWNTs and metal precursors, according to some embodiments.

FIGS. 2A, 2B, 2C and 2D are powder XRD patterns of (A) MWNTs, (B) Pd-MWNTs, (C) Au-MWNTs and (D) Ag-MWNTs, according to the examples.

FIGS. 3A, 3B, and 3C are transmission electron microscopy (TEM) and high-resolution transmission electron microscopy (HRTEM) images of nanocomposites (A) Pd-MWNT, (B) Au-MWNT and (C) Ag-MWNT, according to the examples.

FIGS. 4A, 4B, 4C, and 4D are FTIR spectra of (A) MWNTs, (B) Pd-MWNT, (C) Au-MWNT and (D) Ag-MWNT, according to the examples.

FIG. 5 is a graph illustrating the dependence of thermal conductivity versus volume fraction of MWNT, Pd-MWNT, Au-MWNT, and Ag-MWNT in base fluids of (A) DI water and (B) ethylene glycol, according to the examples.

FIG. 6 is a graph illustrating the temperature dependence of thermal conductivity of (A) Pd-MWNT, (B) Au-MWNT and (C) Ag-MWNT in base fluids of deionized water and ethylene glycol, according to the examples.

### DETAILED DESCRIPTION

In the following detailed description, reference is made to the accompanying drawings, which form a part hereof. The illustrative embodiments described in the detailed description, drawings and claims are not meant to be limiting. Other embodiments may be utilized, and other changes may be made, without departing from the spirit or scope of the subject matter presented here. The present technology is also illustrated by the examples herein, which should not be construed as limiting in any way.

In one aspect, compositions including metal nanoparticles dispersed on multi-walled carbon nanotubes (MWNTs) are provided. Such materials are referred to as metal-MWNTs. In one aspect, the metal nanoparticles are bound to the multi-walled nanotubes. In some embodiments, the metal nanoparticles are bound to the multi-walled nanotubes through functional groups on a surface of the multi-walled nanotube.

The metals that may be used in the present technology include all metal compounds that are capable of being bound to the multi-walled nanotubes. Non-limiting examples of such metals may include, but are not limited to, transition metals such as Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg or Ac. In some embodiments, the transition metal is Au, Ag, Pd, Pt, Cu, Ni, Co, Rh, or Ir.

The functional groups used in the present technology include any suitable group which is capable of bonding the metal to the surface of the multi-walled nanotube. In some embodiments, the functional group is a hydrophilic functional group. In some embodiments, the functional groups include those which contain a hydrophilic oxygen, such as, e.g., a carboxylate, hydroxyl or carbonyl group.

The metal nanoparticles partially coat the surface of the MWNTs by binding to the functional groups as described above. The coating, however, is described in terms of loading, i.e. how much of the metal nanoparticles are incorporated with the MWNTs by weight. According to some embodiments, the MWNTs are loaded with the metal nanoparticles from 0.1 to 30 wt %. In other embodiments, the, the MWNTs are loaded with the metal nanoparticles from 5 to 25 wt %. In other embodiments, the, the MWNTs are loaded with the metal nanoparticles from 10 to 20 wt %. In other embodiments, the, the MWNTs are loaded with the metal nanoparticles at about 20 wt %.

The metal-MWNTs of the present technology can be structurally and morphologically characterized through the use of X-ray diffraction analysis (XRD), high resolution transmission electron microscopy (HRTEM), energy dispersive X-ray analysis (EDX) and Fourier transform infrared spectroscopy (FTIR).

In one aspect, a nanofluid is provided which includes a composition containing metal nanoparticles bound to MWNTs through functional groups on the surface of the nanotube dispersed in a base fluid. The compositions may be dispersed in a fluid, with or without the use of surfactants or dispersants, to form a nanofluid. Dispersion is determined by non-settlement of the metal nanoparticle—MWNTs after a predetermined amount of time. For example, in some embodiments, the non-settlement is after 1 minute. In other embodiments, the non-settlement is after 10 minutes. In other embodiments, the non-settlement is after 1 hour. In yet other embodiments, the non-settlement is after 24 hours. In yet further embodiments, the non-settlement is after 1 week. In some embodiments, the compositions are dispersed in a base fluid, however such base fluids do not include a surfactant. In some embodiments, the metal-MWNTs are loaded in the base fluids at a volume fraction of 0.005% to 0.03%. In other embodiments, the metal-MWNTs are loaded in the base fluids at a volume fraction of 0.01% to 0.03%.

As used herein, “surfactants” are materials which lower the surface tension of a liquid, allowing for a lowering of the interfacial tension between a liquid and a solid. In some embodiments, surfactants include, but are not limited to sodium lauryl sulfate, oleic acid, and gum arabic.

The base fluid includes any fluid which exhibits thermal conductivity. Such fluids include, but are not limited to, water, ethylene glycols, tri-ethylene glycols, oils, and other conventional base fluids. In some embodiments, the base fluid is water. In some embodiments, the base fluid is de-ionized water (DI-water). In other embodiments, the base fluid is ethylene glycol (EG).

The nanofluids of the present technology, which include the composition containing metal nanoparticles bound to MWNTs, exhibit improved thermal conductivity over base fluids without the composition. Thus, in some embodiments, the nanofluid exhibits a thermal conductivity that is at least 2% greater than the base fluid without the composition. In other embodiments, the nanofluid exhibits a thermal conductivity that is at least 5% greater than the base fluid without the composition. In further embodiments, the nanofluid exhibits a thermal conductivity that is at least 10% greater than the base fluid without the composition. In yet further embodiments, the nanofluid exhibits a thermal conductivity that is at least 25% greater than the base fluid without the composition. In some other embodiments, the nanofluid exhibits a thermal conductivity that is at least 50% greater than the base fluid without the composition. In some other embodiments, the nanofluid exhibits a thermal conductivity that is from 5% to 50% greater than the base fluid without the composition. In some other embodiments, the nanofluid exhibits a thermal conductivity that is from 25% to 50% greater than the base fluid without the composition. Temperature dependence studies of thermal conductivity of nanofluids, which include the compositions of the present technology, exhibit an improvement or enhancement of thermal conductivity with temperature. For example, a maximum enhancement of 37% and 11% in thermal conductivity was observed in Ag-MWNTs nanofluid, using DI water and EG as base fluids, respectively, at a volume fraction of 0.03%.

In another aspect, methods for preparing the metal-MWNT compositions are provided. In some embodiments, the method includes refluxing the multi-walled nanotubes in concentrated acid to form a multi-walled nanotube which includes functional groups, collecting the multi-walled nanotubes which include functional groups, and reacting the multi-walled nanotube comprising the functional groups, a metal salt, and a reductant in a solvent to form the multi-walled nanotubes which include metal nanoparticles (metal-MWNTs).

The MWNTs used in the present methods can be synthesized or purchased commercially. The MWNTs are refluxed with a suitable acid, e.g., a concentrated acid to form a MWNT which has functional groups. The concentrated acid used in the methods include any acid capable of forming a functional group on the MWNT surface can be used. In some embodiments, the acid is sulfuric acid or nitric acid. Depending on the type of acid, the functional group may include a carboxylate, hydroxyl or carbonyl group. Without being bound to a theory, it is believed that the acid refluxing step makes the MWNTs hydrophilic by oxidizing the stable aromatic rings of the MWNT and attaching the hydrophilic oxygen-containing functional groups at the surface of the MWNTs. The MWNTs which include functional groups can then be washed with water to remove excess acid. In some embodiments, the MWNTs, which include functional groups, are washed with water until the wash liquid registers a neutral pH. The MWNTs, which include functional groups, are then collected by filtration. Functional groups present on the MWNT surfaces not only impart hydrophilic character, but also act as anchoring sites for metal nanoparticles.

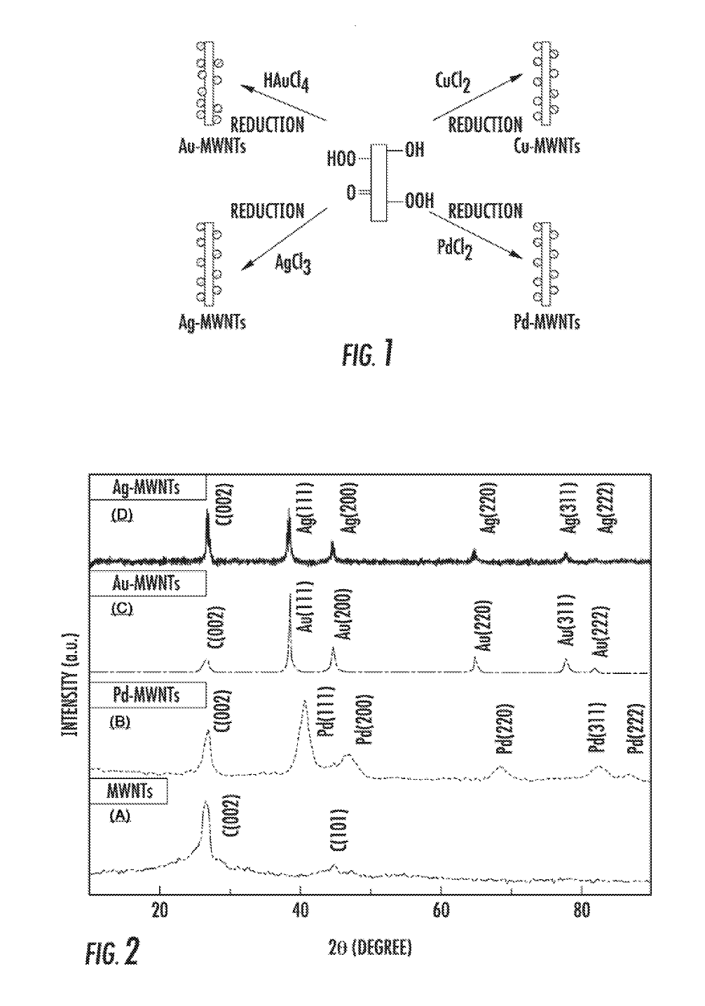

In some embodiments, the metal coated MWNTs can be prepared by chemical reduction methods using metal salt precursor and functionalized MWNTs, whereby the metal is bound to the MWNTs as seen in FIG. 1. It is believed that the attached functional groups such as, e.g., hydroxyl, carboxyl or carbonyl groups provide nucleation sites for the metals to bind to the surface of the MWNTs. The metals used in the methods include all metal compounds that are capable of being bound to the multi-walled nanotubes. Non-limiting examples of such metals may include transition metals such as, e.g., Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg or Ac. In some embodiments, the transition metal is Au, Ag, Pd, Pt, Cu, Ni, Co, Rh, or Ir.

The metals may be used in their salt forms. In illustrative embodiments, salts include the halides, e.g., the chlorides, bromides and fluorides, including halide hydrates; acetates, oxides, oxyhalides, nitrates, cyanates, thiocyanates, chlorates, phosphates, sulfates, carbonates, bromates, chromates, cyanides, ferrocyanides, fluoborates, iodates, nitrites and sulfites and combinations thereof. In some embodiments, the metal salts include F⁻; Cl⁻; Br⁻; I⁻; NO₃⁻; CN⁻; ClO₄⁻; or CH₃COO⁻. The metal precursor is not particularly limited to the above metals or metal salts and may be any metal reducible by the reductant.

The chemical reduction reaction between functionalized MWNTs and metal salts can be conducted by any suitable reduction methods known in the art. The reductant or reducing agent for reducing the metal salt is not particularly limited. Known reductants including, but not limited to, gases such as H₂, NH₃ and CO, H₂-plasma, light irradiation, hydrazines, citrates, and borohydrides, can be used in the present methods. Illustrative reducing agents include, but are not limited to, sodium borohydride (NaBH₄), potassium borohydride (KBH₄), lithium aluminum hydride (LiAlH₃); alcohols such as glycol, ethanol, propanol, and isopropanol; hydrogen iodide (HI); and secondary or tertiary amine compounds such as hydroxylamine, hydrazine compounds, dimethylaminoethanol, and dimethylethylamine. In some embodiments, the reductant is sodium borohydride (NaBH₄). The concentration of the reductant, the reduction time, the reduction temperature, etc., can be deduced from known reduction methods. In some embodiments, an alkali, e.g., NaOH, may also be added during the reducing step.

The solvent used in the methods is not particularly limited so long as the MWNTs can be efficiently dispersed therein and the metal ion-containing compound (such as a metal salt and a metal complex compound) can be dissolved therein to generate a metal nanoparticle. Thus, solvents useful in the present methods may include, but are not limited to, polar organic solvents, including but not limited to, ethylene glycol, acetone, tetrahydrofuran, dimethyl formamide, dimethyl sulfoxides and dimethyl sulfate; nonpolar organic solvents including but not limited to, pentane, hexane, heptane, and octane; alcohols including but not limited to, methanol, ethanol, n-propanol, iso-propanol, butanols, and pentanols; halogen-substituted organic solvents including but not limited to, chloroform, methylene chloride, and carbon tetrachloride; and water. For example, the solvent may include water, acetone, toluene, tetrahydrofuran, methanol, ethanol, chloroform, hexane, dimethyl formamide, dimethyl sulfoxide or dimethyl phosphate. In some embodiments, the functionalized MWNTs are dispersed into the solvent using ultrasonication prior to their interaction with the metal compound.

In yet another aspect, methods for preparing nanofluids are provided. In some embodiments, the method includes sonicating the multi-walled nanotube comprising metal nanoparticles in a base fluid. Depending upon the type and the quantity of base fluid used, the process may be conducted using an ultrasonicator of sufficient strength and for a suitable amount of time to effectively disperse the metal-MWNTs in the base fluid. For example, the sonication may take place from 10 minutes to 1 hour, from 15 minutes to 1 hour, from 30 minutes to 1 hour, or from 40 to 50 minutes. Without being bound to a theory, it is believed that the ultrasound produces microscopic bubbles in the liquid. These bubbles, when collapsed, result in shock waves which are highly effective in increasing the nanotube hydrophilicity and result in dispersing metal-MWNTs homogenously in the base fluid.

The base fluid includes any fluid which exhibits thermal conductivity. Such fluids include, but are not limited to, water, ethylene glycols, tri-ethylene glycols, oils, and other conventional base fluids. In some embodiments, the base fluid is water. In some embodiments, the base fluid is de-ionized water (DI-water). In other embodiments, the base fluid is ethylene glycol (EG). The metals used in the methods include all metal compounds that are capable of being bound to the multi-walled nanotubes. Non-limiting examples of such metals may include transition metals such as, e.g., Sc, Ti, V, Cr, Mn, Fe, Co, Ni, Cu, Zn, Y, Zr, Nb, Mo, Tc, Ru, Rh, Pd, Ag, Cd, La, Hf, Ta, W, Re, Os, Ir, Pt, Au, Hg or Ac. In some embodiments, the transition metal is Au, Ag, Pd, Pt, Cu, Ni, Co, Rh, or Ir. In some embodiments, the nanofluid includes multi-walled nanotubes with Pd, Au, or Ag nanoparticles; and the base fluid includes water or ethylene glycol.

The nanotubes used in the present methods and compositions can be synthesized using standard methods known in the art. For example, the MWNTs can be synthesized by the decomposition of acetylene on hydrogen-decrepitated YNi₃ alloy hydride particles using single furnace chemical vapor depositions (CVD) technique. As used herein, “decrepitated” refers to the roasting or calcining of a substance until a crackling sound stops. Hydrogen-decrepitated refers to the roasting or calcining under a hydrogen atmosphere. The prepared nanotubes can then be used as such, or subjected to further purification. Standard purification methods known in the art such as air oxidation, and acid treatment, can be used to purify the nanotubes. Purified MWNTs may be functionalized, decorated with metals, and reduced as described above to improve the dispersion of MWNTs in base fluids. Because the metal-MWNTs of the present technology possess hydrophilic character, they are readily dispersed in the base fluid, even without the use of surfactants.

Thermal conductivity measurements for the nanofluids may be performed using standard methods known in the art, such as, but not limited to, hot wire methods or Differential Scanning Calorimetry (DSC). The nanofluids provide high thermal conductivity/heat transfer even at low volume fraction of less than 0.03% without the use of surfactants and eliminate, or at least exhibit reduced settlement or aggregation of nanotubes. The dispersion of a small amount of the metal-MWNTs in a base fluid leads to large enhancement in thermal conductivity of the suspension.

One skilled in the art will readily realize that all ranges discussed can and do necessarily also describe all subranges therein for all purposes, and that all such subranges also form part and parcel of this disclosure. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

The present technology, thus generally described, will be understood more readily by reference to the following examples, which are provided by way of illustration and are not intended to be limiting.

### EXAMPLES

The present technology is further illustrated by the following examples, which should not be construed as limiting in any way.

### Example 1

### Synthesis of MWNTs

MWNTs have been synthesized through the decomposition of acetylene on hydrogen decrepitated YNi₃ alloy hydride particles using single furnace chemical vapor deposition (CVD). The CNTs were purified using by oxidization in air, followed by acid treatment.

The purified MWNTs were then functionalized for the dispersion of MWNTs in polar solvents by refluxing the MWNTs in highly concentrated acid for 12 h to oxidize the stable aromatic rings and attach hydrophilic oxygen containing functional groups (—COOH, —C═O and —OH) at the surface. The sample was then washed several times with copious amounts of water until the pH was registered neutral.

### Example 2

### Synthesis of Metal-Coated MWNTs

Metal nanoparticle-coated MWNTs nanocomposites were prepared by chemical reduction using a metal salt precursor and a functionalized MWNT, in the presence of a reductant. The metal is dispersed on the MWNTs prepared according to Example 1. The hydroxyl, carboxyl and carbonyl groups attached to the surface of the functionalized MWNTs provide nucleation sites for the metal nanoparticles. Purified MWNTs (0.2 g) were ultra-sonicated in acetone (20 ml) for 1 h, followed by addition of the metal salt solution and stirring for 24 h. The salt was then reduced by the slow addition of a solution of 0.1 M NaBH₄ and 1 M NaOH. Once the reaction was complete, the solution was washed with DI water, filtered, and dried at 80° C. for 3 h.

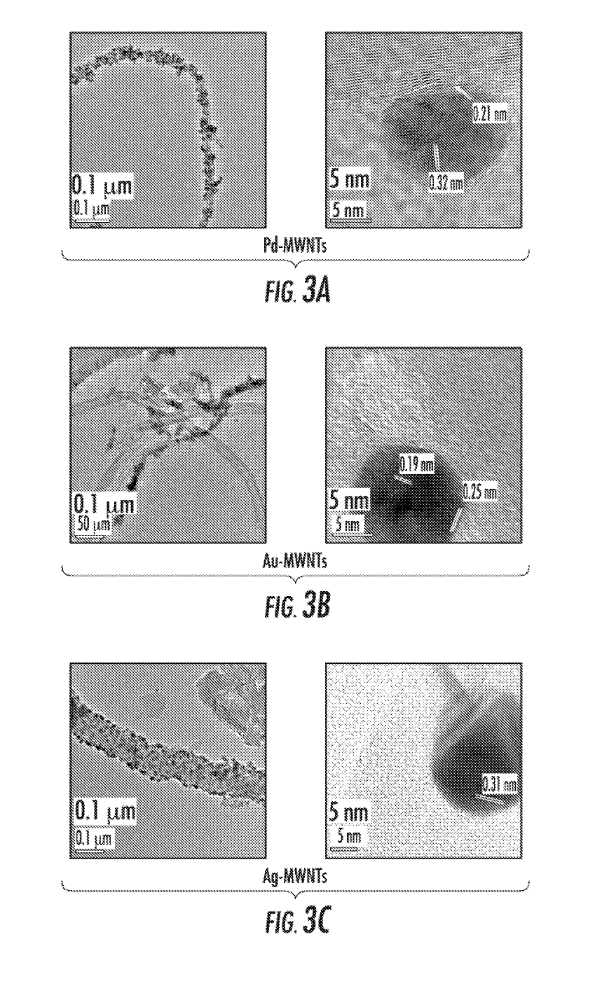

The metal-coated MWNTs were characterized by powder XRD (Panalytical X-Pro) at room temperature. FIG. 2 shows XRD patterns of MWNTs and metal-MWNTs. FIG. 2A shows reflections at 2θ values 26 and 44 degrees corresponding to graphitic crystalline planes C(002) and C(101), respectively, of MWNTs. It also confirms hexagonal arrangement of carbon atoms in the MWNTs. In the case of the Pd-MWNTs, peaks appear at 2θ values 40, 46, 68, 82 and 87 corresponding to fcc crystalline reflections from Pd nanoparticles planes (111), (200), (220), (311) and (222), respectively, with peaks from MWNTs, which indicates the presence of both Pd nanoparticles as well as MWNTs in the nanocomposite. Similarly, in the case of Au-MWNTs, peaks appear at 2θ values of 38, 44, 64, 77 and 82 corresponding to fcc Au planes (111), (200), (220), (311) and (222), respectively. Peaks corresponding to the fcc Ag-MWNTs appear at 2θ values of 38, 44, 64, 77 and 81, and are indexed at (111), (200), (220), (311) and (222), respectively, along with the peaks corresponding to MWNTs.

High resolution TEM (JEOL 3010 coupled to a Gatan digital camera) images of the metal-MWNTs are shown in FIG. 3, which confirms the homogenous dispersion of metal nanoparticles on the outer surface of MWNTs. The lattice planes of metal nanoparticles are observed in the HRTEM images indicating their crystallinity. Efficient and homogenous dispersion of metal particles in the size range of from 10 nm to 20 nm on the MWNTs surface has been observed. EDX analysis of the metal-MWNTs confirms the presence of metal particles as well as carbon in the nanocomposite.

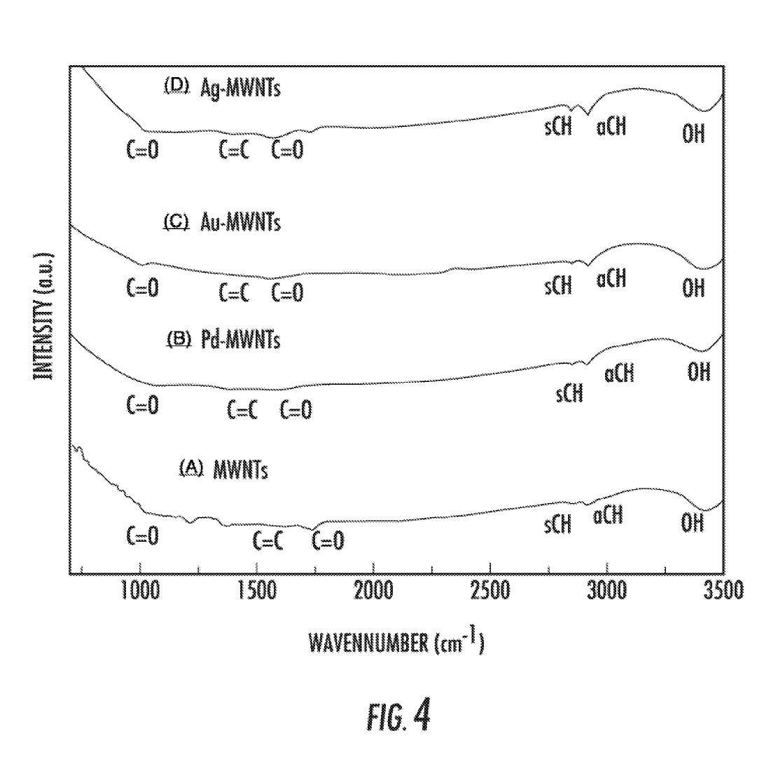

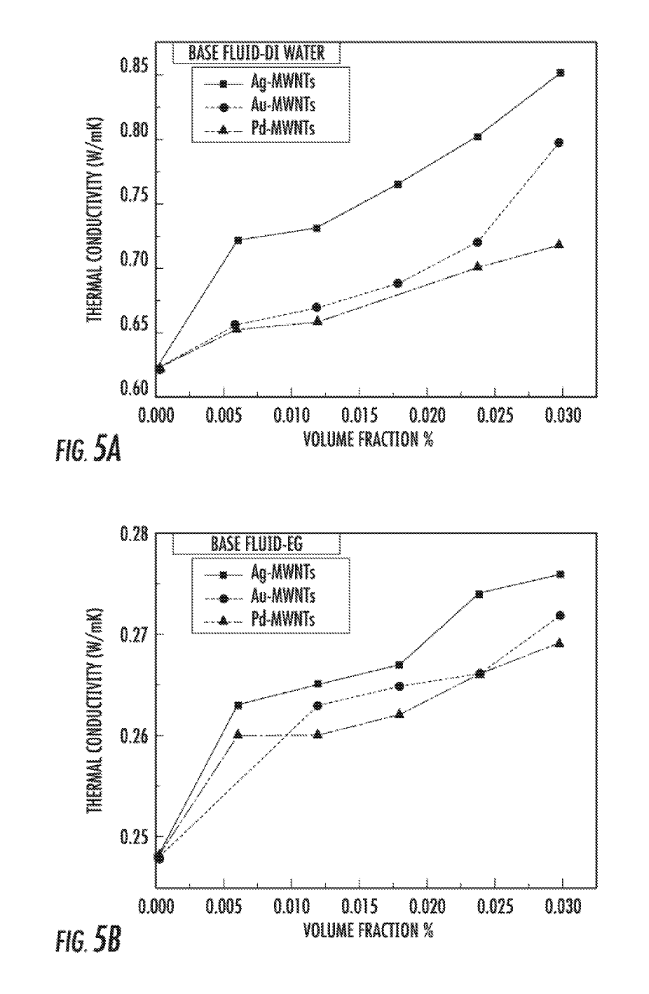

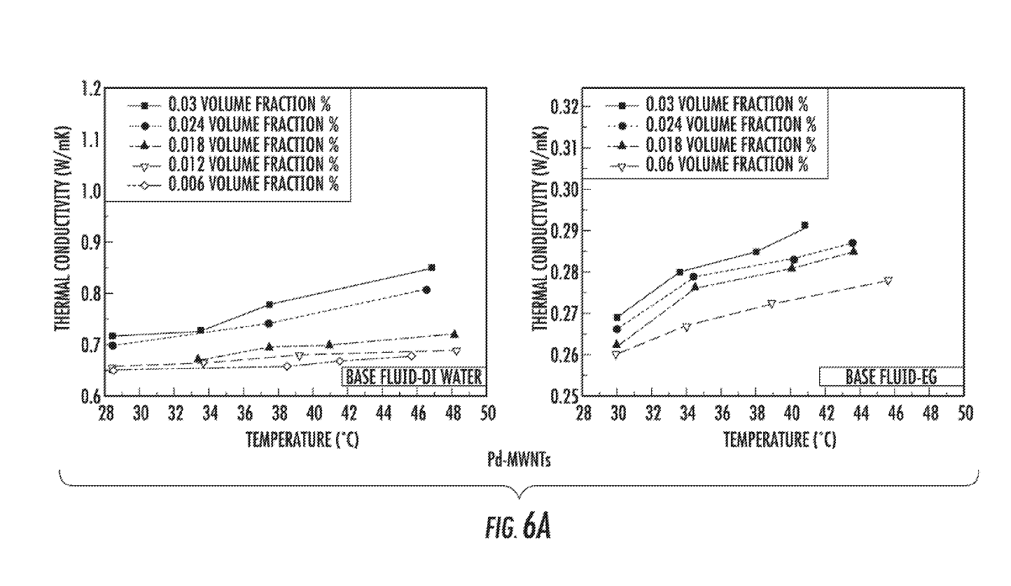

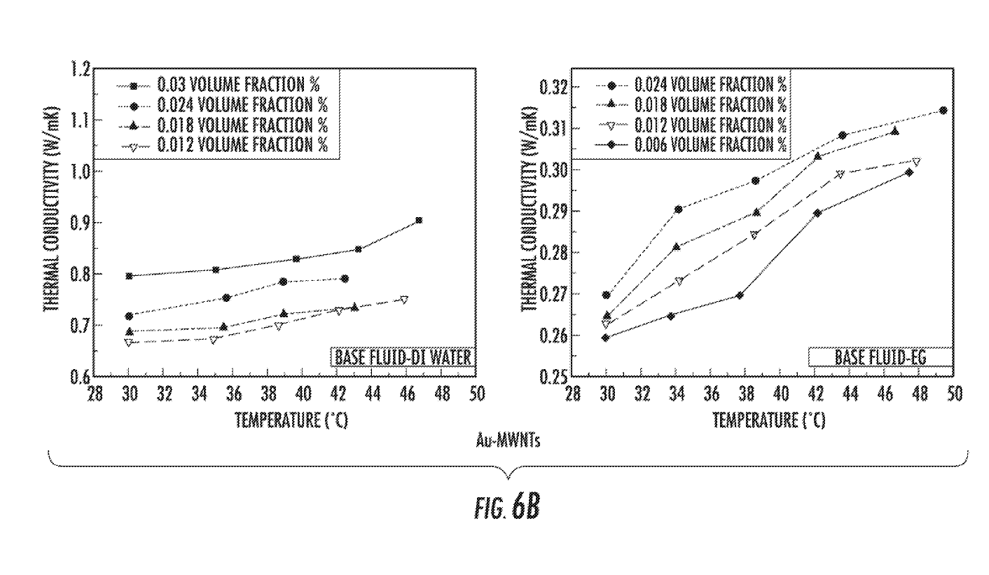

FIGS. 4A-4D show FTIR spectra of MWNTs, Pd-MWNTs, Au-MWNTs and Ag-MWNTs, respectively. All of the metal-MWNTs samples show peaks around 3420 cm⁻¹, which corresponds to the hydroxyl group (—OH). This peak can be attributed to —OH functional groups attached to the surface of MWNTs. Bands due to asymmetric and symmetric stretching of CH bonds are centered around 2840 and 2920 cm⁻¹. The peak centered around 1720 cm⁻¹ is assigned to the C═O stretching vibration in the —COOH group. Peaks around 1560 cm⁻¹ in all the samples are due to aromatic C═C vibration. Bands around 1086 cm⁻¹ are assigned to C—O bond stretching. Thus, the presence of —OH and —COOH groups on all the metal-MWNTs samples were observed, even after the attachment of metal nanoparticles, which aids in the dispersion of the samples in DI water and EG base fluids without the addition of any surfactant.

### Example 3

### Preparation of Nanofluid

The metal-coated MWNTs are dispersed in a base fluid. The calculated amount of the metal-coated MWNTs was placed in a sample bottle with the base fluid of choice. The sample bottle and contents were then sonicated for about 40 min using a 100 W, 40 kHz ultrasonicator. The ultrasound produces microscopic bubbles in the liquid which then collapse and produce shock waves in the liquid. The shock waves are effective in dispersing metal-MWNTs homogenously in the base fluid. Nanofluids prepared by such methods include Pd-MWNTs/DI water, Au-MWNTs/DI water, Ag-MWNTs/DI water, Pd-MWNTs/EG, Au-MWNTs/EG, and Ag-MWNTs/EG.

### Example 4

### Measurement of Thermal Conductivity

The thermal conductivity of the suspension was measured using a KD2 Pro Thermometer (Decagon, Canada). The KD2 meter is based on a transient hot wire method having a probe of length 6 cm and 0.09 cm diameter. This probe integrates in the interior, and consists of a heating element and a thermo-resistor connected to a microprocessor for controlling as well as conducting measurements. The KD2 meter was calibrated using distilled water before use. To study the temperature effect on the thermal conductivity of the nanofluids, a thermostat bath was used, which maintained temperature within the range of ±0.02° C. Five measurements were taken at each temperature to ensure uncertainty in the measurement is within ±5%.

The thermal conductivity of the fluids has been measured and is presented in Table 1. Table 1 also provides comparison data to the pure metal particles and base fluids.

TABLE I
Thermal Conductivity data for nanoparticles and nanofluids

| | Thermal | | % increase in |
|---|---|---|---|
| | Conductivity | Volume | Thermal |
| Nanoparticles/Nanofluid | (W/m K) | Fraction % | Conductivity |
| MWNTs | 3000 | — | — |
| Palladium | 71.8 | — | — |
| Gold | 317 | — | — |
| Silver | 429 | — | — |
| DI-Water | 0.62 | — | — |
| MWNTs/DI Water | 0.712 | 0.04 | 14.8 |
| Pd-MWNTs/DI Water | 0.718 | 0.03 | 15.8 |
| Au-MWNTs/DI Water | 0.773 | 0.03 | 28.1 |
| Ag-MWNTs/DI Water | 0.851 | 0.03 | 37.3 |
| EG | 0.248 | — | — |
| MWNTs/EG | 0.266 | 0.04 | 7.3 |
| Pd-MWNTs/EG | 0.268 | 0.03 | 8.5 |
| Au-MWNTs/EG | 0.272 | 0.03 | 9.7 |
| Ag-MWNTs/EG | 0.276 | 0.03 | 11.3 |

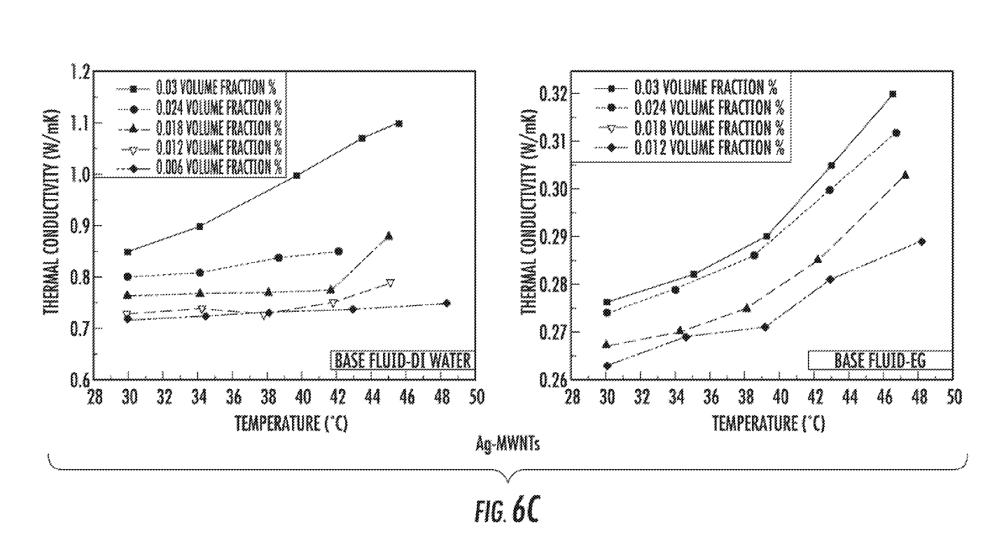

As seen in Table 1, for Pd-MWNTs nanofluids with DI water and EG base fluids, enhancement in thermal conductivity of about 24% and 8% has been obtained at a volume fraction of 0.03%, which is higher compared to pure MWNTs nanofluid. In the case of Au-MWNT nanofluids, an enhancement of 28% is obtained with DI water as a base fluid and about 9.7% for ethylene glycol base fluid. In the case of Ag-MWNT nanofluids, 37 and 11% increase in thermal conductivity with DI water and EG base fluids, respectively, which are achieved at the volume fraction of 0.03% as seen in FIG. 5. The enhancement of the thermal conductivity of a nanofluid is attributed to the increase in the thermal conductivity of dispersed nanocomposite. The increase in specific surface area of the dispersed nanocomposite, which, in turn, increases the effective base fluid-nanoparticle interfacial layer, leads to the enhancement in the amount of heat transfer.

It has been observed that metal-MWNTs nanofluids with DI water and EG base fluids maintain the same sequence of thermal conductivity as of the nanoparticles, i.e., Ag-MWNTs>Au-MWNTs>Pd-MWNTs (Table I). In addition, in the present study, the percentage increase in thermal conductivity of DI water-based nanofluid is greater in comparison to EG-based nanofluid with the same volume fraction of metal-MWNTs which is due to the ease of transfer of thermal energy in DI water. Hence, the metal nanoparticles decorated MWNTs based nanofluids are suitable candidates for enhancing thermal transport in heat transfer applications.

Temperature dependence of the thermal conductivity of the Au-MWNTs, Ag-MWNTs and Pd-MWNTs nanofluids was studied at different concentrations. FIG. 6 shows an enhancement of 37 and 17% at high temperatures for Pd-MWNTs nanofluids in DI Water and EG base fluids, respectively. Similarly, in the case of Au-MWNTs, 45 and 27% enhancement has been observed at high temperature with DI water and EG base fluid, respectively. Ag-MWNTs nanofluid also show similar results with 77 and 29% increase in the thermal conductivity with DI water and EG, respectively. It has been found that thermal conductivity increases linearly with temperature indicating that the Brownian motion of the nanoparticles suspended in the nanofluid plays a major role in the heat transfer.

### EQUIVALENTS

While certain embodiments have been illustrated and described, it should be understood that changes and modifications can be made therein in accordance with ordinary skill in the art without departing from the technology in its broader aspects as defined in the following claims.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art, all language such as “up to,” “at least,” “greater than,” “less than,” and the like, include the number recited and refer to ranges which can be subsequently broken down into subranges as discussed above. Finally, as will be understood by one skilled in the art, a range includes each individual member.

Other embodiments are set forth in the following claims.

What is claimed is:

1. A composition comprising:
a multi-walled nanotube comprising metal nanoparticles;
wherein:
the metal nanoparticles are bound to the multi-walled nanotube through functional groups on a surface of the multi-walled nanotube.

2. The composition of claim 1, wherein the metal nanoparticle comprises a transition metal.

3. The composition of claim 2, wherein the transition metal is selected from the group consisting of Au, Ag, Pd, Pt, Cu, Ni, Co, Rh, and Ir.

4. The composition of claim 1, wherein the functional group is a carboxylate, hydroxyl, or carbonyl group.

5. A nanofluid comprising the composition of claim 1 dispersed in a base fluid, with the proviso that the nanofluid does not comprise a surfactant or dispersion agent.

6. The nanofluid of claim 5, wherein the base fluid is water.

7. The nanofluid of claim 6, wherein the base fluid is de-ionized water.

8. The nanofluid of claim 5, wherein the base fluid is ethylene glycol.

9. The nanofluid of claim 5, wherein the nanofluid exhibits a thermal conductivity that is at least 5% greater than the base fluid without the composition.

10. The nanofluid of claim 5, wherein the nanofluid exhibits a thermal conductivity that is at least 25% greater than the base fluid without the composition.

11. A nanofluid comprising:
a multi-walled nanotube coated with Pd, Au, or Ag; and
a base fluid that is water or ethylene glycol;
with the proviso that the nanofluid does not comprise a surfactant or dispersion agent.

12. A method of preparing the composition of claim 1, comprising:
refluxing the multi-walled nanotube in concentrated acid to form a multi-walled nanotube comprising functional groups;
collecting the multi-walled nanotube comprising functional groups;
reacting the multi-walled nanotube comprising functional groups, a metal salt, and a reductant in a solvent to form the multi-walled nanotube comprising metal nanoparticles.

13. The method of claim 12, wherein the metal salt is a transition metal salt.

14. The method of claim 13, where the transition metal salt comprises a metal selected from the group consisting of Au, Ag, Pd, Pt, Cu, Ni, Co, Rh, and Ir.

15. The method of claim 12, wherein the metal salt comprises F⁻; Cl⁻; Br⁻; I⁻; NO₃⁻; CN⁻; ClO₄⁻; or CH₃COO⁻.

16. The method of claim 12, wherein the reductant is NaBH₄.

17. The method of claim 12, wherein the solvent is acetone.

18. A method of preparing the nanofluid of claim 4, comprising:
sonicating the multi-walled nanotube comprising metal nanoparticles in a base fluid.

19. The method of claim 18, wherein the base fluid comprises water or ethylene glycol.

20. The method of claim 18, wherein the nanofluid comprises:
multi-walled nanotubes comprising Pd, Au, or Ag nanoparticles; and
the base fluid comprises water or ethylene glycol.
